(19) United States
(12) Patent Application Publication
(10) Pub. No.: US 2002/0151458 A1
(43) Pub. Date: Oct. 17, 2002

(54) Method for treating and preventing septic shock with VPAC1R, VPAC2R and PAC1R agonists

(76) Inventors: Rosa Maria Perez Gomariz, Madrid (ES); Javier Leceta Martinez, Madrid (ES); Mario Delgado Mora, Madrid (ES); Carmen Martinez Mora, Madrid (ES)

Correspondence Address: NOTARO AND MICHALOS, 100 DUTCH HILL ROAD, SUITE 110, ORANGEBURG, NY, 10962-2100, US

(21) Appl. No.: 10/090,109

(22) Filed: Mar. 4, 2002

Related U.S. Application Data
(63) Continuation-in-part of application No. 09/446,352, filed on Apr. 3, 2000, now Pat. No. 6,429,188.
Application No. 09/446,352 is a 371 of international application No. PCT/ES99/00101, filed on Apr. 16, 1999.

(30) Foreign Application Priority Data
Apr. 17, 1998 (ES) P9800814

(51) Int. Cl. A61K 31/00

(52) U.S. Cl. 514/1

(57) ABSTRACT

A pharmaceutical composition for the treatment and/or prevention of septic shock has VPAC1, VPAC2 or PAC1 receptor agonist combined with a pharmaceutically acceptable carrier. A method for treating or preventing septic shock is provided as well in which a therapeutically effective amount of the pharmaceutical composition is administered to a patient.

### CROSS-REFERENCE TO RELATED APPLICATIONS

This application is a continuation-in-part application of pending U.S. patent application Ser. No. 09/446,352, which is hereby incorporated by reference in its entirety.

### FIELD AND BACKGROUND OF THE INVENTION

The present invention relates generally to the field of immunology and control of host defense mechanisms. More specifically, this invention relates to the use of VPAC1R, VPAC2R, and PAC1R agonists, that can be administered in therapeutically effective doses to treat and/or prevent septic shock.

Despite recent progress in antibiotics and critical care therapy, sepsis is still associated with a high mortality rate. Septic shock and sequential multiple organ failure/multiple organ dysfunction syndrome (MOF/MODS) correlate with poor outcome.

Lipopolysaccharide (LPS) or endotoxin plays a pivotal role in the initiation of a variety of host responses caused by Gram-negative bacterial infection (See, J. E. Parrillo, New England Journal of Medicine 1993, 328:1471). Since the 1980s, novel insights into the molecular pathogenesis of LPS-induced shock (endotoxic shock) and organ dysfunction have been gained. The molecular cloning of proinflammatory cytokines and adhesion molecules were important steps towards understanding the molecular mechanism of sepsis. In addition, nitric oxide (NO) has been identified as a key mediator of LPS-mediated hypotension (See, L. L. Moldawer, Critical Care Medicine 1994, 22:3).

In 1991, a new concept known as Systemic Inflammatory Response Syndrome (SIRS) was postulated to define the state of patients who exhibit a systemic response to inflammatory episodes. SIRS is diagnosed by a combination of available clinical signs and symptoms. Currently, sepsis is generally defined as SIRS induced by infection.

No world-wide statistics on the occurrence of sepsis are available. In the United States alone, however, it is estimated that there are 300,000-500,000 septic episodes each year, with mortality rates ranging from 20% to 40%. Refractory hypotension (septic shock) is the main cause of death within a few days of the onset of sepsis. Later, MOF/MODS becomes the primary clinical problem and main cause of mortality. Once a patient develops septic shock or sequential MOF/MODS, the mortality rate increases to 60-70%.

Gram-negative bacteria are responsible for 45-60% of sepsis caused by bacterial infection when mixed-organism infections are included (See, R. Karima et al., Molecular Medicine Today 1999, 5:123). A variety of pathophysiological responses in various tissues and organ systems occur during endotoxemia. In particular, circulatory failure, leukocyte-induced tissue injury and activation of coagulation systems appear to be critical determinants in the development of sequential organ failure.

A number of mediators derived from host cells are responsible for most of the manifestations of endotoxemia. The proinflammatory cytokines, including tumor necrosis factor alpha (TNFα), interleukin (IL) 1β, IL-6, IL-8 and IL-12, and interferon gamma (IFNβ), play a critical role in the inflammatory responses. Nitric oxide (NO) is now known to induce a variety of responses in addition to hypotension.

Furthermore, anti-inflammatory mediators, such as IL-10 and IL-1 receptor antagonist (IL-1Ra), also contribute to the modulation of inflammatory responses in endotoxemia.

TNFα is produced by several types of cells that include monocytes and macrophages, T and B lymphocytes, neutrophils, mast cells, tumorous cells, and fibroblasts. It is an important regulatory factor in other pro-inflammatory cytokines, such as IL-1β, IL-6, and IL-8. TNFα induces the expression of adhesion molecules in endothelial cells, activates leukocytes to destroy the microorganisms, acts on the hepatocytes to increase the systhesis of serum proteins which contribute to the acute phase response and activate the coagulation system. Overproduction thereof leads to immunopathologic diseases, autoimmunity and inflammation.

IL-6 is a multi-functional cytokine produced both by lymphocytes and non-lymphoid cells. It regulates several aspects of the immune response, such as the production of proteins that mediate acute phase and hematopoiesis. Furthermore, it acts as a mediator in inflammatory response. Its production is regulated by several factors, which include TNFα, IL-1, and LPS.

Nitric oxide (NO) is an unstable free radical gas that mediates many physiological and toxic functions, such as macrophage cytotoxicity, neurotransmission, neurotoxicity, and regulation of blood pressure. Induced NO production is one of the principal mechanisms of macrophage cytotoxicity for tumor cells, bacteria, protozoa, helminthes and fungi.

In general, production of NO follows a generalized or localized inflammatory response resulting from infection or tissue injury. Despite its beneficial role in host defense, sustained NO production can be deleterious to the host. Nitric oxide synthesis induced by cytokines and/or inflammatory stimuli has been implicated in experimental arthritis, inflammatory bowel disease, hypotension associated with septic shock, and other types of tissue injury.

IL-12, another early proinflammatory cytokine secreted by macrophages activated by microbial products, plays a central role in the regulation of cell-mediated immunity. IL-12 stimulates the proliferation of activated T lymphocytes and enhances IFNγ secretion by NK cells and T lymphocytes. Consistent with the latter effect, IL-12 has a pivotal role in the induction of CD4⁺ Th1 cell responses, acting in antagonism to IL-4, the major promoter of the Th2 response. In mice, IL-12 plays a decisive role in the protection against intracellular pathogens, including parasites and bacteria.

However, the central importance of IL-12 and IFNY in the pathogenesis of the endotoxic shock is indicated by the fact that pretreatment with corresponding neutralizing antibodies protects against lethality.

IL-10, one of the major anti-inflammatory cytokines, was initially described as a Th2 product that inhibits the secretion of Th1-derived cytokines, through the down-regulation of the antigen-presenting function of professional antigen presenting cells. In addition to T cells, activated monocytes/macrophages serve as a major IL-10 source, especially in response to LPS stimulation. IL-10 inhibits several macrophage functions, such as oxidative burst, phagocytosis, nitric oxide production, and cytokine production. Administration of IL-10 to endotoxic mice showed protective effects against proinflammatory cytokine production and lethality.

Strategies of neutralization of the pro-inflammatory cytokines have been tested in the treatment of endotoxic shock but the results do not indicate that there is a greater long-term survival (See, G. Zaneti and M-P. Glauser, Current Opinion in Infectious Diseases 1997, 10:139).

A treatment that inhibits the production of different pro-inflammatory cytokines and mediators would represent a considerable improvement in the evolution of endotoxic shock and in the probabilities of survival.

Vasoactive intestinal peptide (VIP) was first isolated from the porcine duodenum and in 1974, Mutt and Said (See, V. Mutt et al., European Journal of Biochemistry 1974, 42:581) established the amino acid sequence. VIP contains 28 amino acid residues, with a highly conserved sequence in vertebrates, a fact that is consistent with its important biological role. It is known today that VIP is a pleiotropic peptide produced by neurons in different areas of the central and peripheral nervous system and by endocrine cells as the pituitary lactotrophes and cells of the endocrine pancreas.

The pituitary adenilate cyclese activating peptide (PACAP) is a member of the family of peptides of the secretin/VIP/glucagon, of which two molecular forms are known, namely PACAP-38 and PACAP-27, whose sequence was determined by Ogi et al (K. Ogi et al., Biochemical and Biophysical Research Comunications 1993, 196:1511). Both peptides are widely distributed in the central and peripheral nervous system.

VIP together with PACAP, secretin and GRF receptors constitute a subfamily based on the homology of both ligands and receptors. To date, three VIP/PACAP receptors have been identified to date that are membrane-bound receptors belonging to the family 2 of G protein-coupled receptors (GPCR).

The six families of (GPCR) have a common central domain constituted of seven transmembrane helices. The family 2 is characterized by a large N-terminal domain which plays an important role in the binding of the ligand, besides for VIP receptors both extracellular and transmembrane domains are also involved. The three VIP/PACAP receptors cloned are: the VPAC1 and VPAC2 that bind VIP and PACAP with equal affinity (See, T. Ishihara et al., Neuron 1992, 8:811; and, A. Covineau et al., Biochemical and Biophysical Research Comunications 1994, 200:769), and the PAC1 receptor that is PACAP selective, although in micromolar amounts, VIP is a heterologous ligand (A. J. Harmar et al., Pharmacological Reviews 1998, 50:265), with eight variants to date produced from alternative splicing of the transcript.

VPAC1, VPAC2, and PAC1 receptors primarily stimulate the adenylate cyclase (AC) pathway. However, it has also been demonstrated only in transfected cell systems, at high expression levels, that VPAC1 may stimulate an inositol triphosphate (IP)/PLC system, as well as cause an increase of intracellular calcium levels.

There is some suggestion that, in a transfected cell system, VPAC2 will also stimulate IP synthesis. However, a clear link to the PLC/IP system remains to be found.

Seven splice variants of the PAC1 receptor are involved in the activation of both AC and IP/PLC systems, and the eighth PAC1 receptor variant, which is also a CGRP receptor, not linked to AC or IP/PLC systems, but that activates an L-type calcium channel.

VPAC1, VPAC2 and PAC1 receptors are expressed in different cell populations in both central nervous system and peripheral tissues.

VPAC1 receptor has been identified in murine isolated thymocytes and T and B lymphocytes from spleen and lymph nodes (See, R. P. Gomariz et al., Biochemical and Biophysical Research Comunications 1994, 203:1599) and also in lymphocytes and macrophages from peritoneal suspensions (See, M. Delgado et al., Regulatory Peptides 1996, 62:161).

VPAC2 receptor was described for the first time as a VIP helodermin-preferring receptor in the human lymphoma cell line SUP T1 (See, P. Roberech et al., Regulatory Peptides 1989, 26:117). VPAC2 expression is inducible in lymphocytes and macrophages. Thus, VPAC2 is detected only following stimulation through the TCR-associated CD3 molecule in lymphocytes or LPS in macrophages (M. Delgado et al., Journal of Neuroimmunology 1996, 68:27).

Moreover, VPAC2 receptor is detected in mononuclear cells by immunohistochemical techniques two days after the detection of VPAC1 at sites of inflammation and antigen recognition (H. B. Kaltreider et al., American Journal of Respiratory Cell and Molecular Biology 1997, 16:133). However, VPAC2 receptor is the only receptor of VIP/PACAP family receptors expressed in some murine T cell lines and in human lymphoid cell lines the constitutive expression of VPAC2 receptor has been reported.

The best characterization of VIP effects exerted through the interaction VPAC1 and VPAC2 in the immune system is the adenylate cyclase pathway.

PAC1 receptor is expressed only in macrophages, as lymphocytes lack PAC1 expression (See, M. Delgado et al., Journal of Neuroimmunology 1996, 68:27; and, D. Pozo et al., Biochemical and Biophysical Acta 1997, 1359: 250). Although PAC1 receptor is the PACAP selective receptor that binds PACAP with greater affinity (from 100-1000 times more) than VIP (See, A. J. Harmar et al., Pharmacological Reviews 1998, 50:265) especially in central nervous system, the PAC1 expressed in freshly isolated macrophages possess similar affinity for both VIP and PACAP (See, D. Pozo et al., Biochemical and Biophysical Acta 1997, 1359: 250-52) and is coupled to the IP/PLC system.

### SUMMARY OF THE INVENTION

It is an object of the present invention to provide a method to treat and/or prevent septic shock in an individual.

It is another object of the invention to provide a treatment that inhibits the production of different pro-inflammatory cytokines and mediators in humans.

A still further object of the present invention is to provide a method for administering a therapeutically effective amount of a VPAC1, VPAC2, or PAC1 receptor agonist together with a pharmaceutically acceptable carrier for treatment of septic shock.

Yet another object of the invention is to provide pharmaceutical compositions containing VPAC1, VPAC2 or PAC receptor agonist for treatment of septic shock in humans.

Accordingly, a method of treating septic shock is provided in which a therapeutically effective amount of a VPAC1, VPAC2, or PAC1 receptor agonist is administered to a person.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 shows the Northern blot analysis for the presence of mRNA corresponding to TNFα and IL-6 in macrophages stimulated with LPS in presence or absence of VIP or PACAP. BALB/c mice were injected with RPMI 1640 medium π: (none), LPS (400 mg), or LPS plus VIP or PACAP-38 (5 nmol);

FIGS. 2A & 2B show the circulating levels of TNFα and IL-6, respectively, in BALB/c mice injected with 400 μg LPS (control), or LPS plus VIP or PACAP-38 (5 nmol). Serum was collected at various time points (from 0 to 12 h), and the levels of TNFαand IL-6 protein were determined by ELISA.

FIGS. 3A & 3B show the survival of BALB/c mice injected with 400 μg of LPS and, either simultaneously or after 30 minutes, 1 or 4 hours, with 5 nmol of VIP or PACAP, respectively.

FIG. 3C shows the survival (%) versus LPS doses injected in mice treated with or without 5 nmol of VIP. Horizontal bars indicate the 95% confidence limits of LD50 determinations.

FIGS. 4A-4E show the production of TNFα, IL-6, IL-12, NO, and IL-10, respectively, by BALB/c mice peritoneal macrophages stimulated with 0.5 μg/ml LPS in the absence (control) or presence of 10-8M VAPC1 or VPAC2 agonists.

FIGS. 5A-5F show the circulating levels of TNFα, IL-6, IL-12, IFNY, IL-10, and NO, respectively, in BALB/c mice injected with 100 μg/mouse LPS (control), LPS plus 5 nmol/mouse VPAC1 agonist, or LPS plus 5 nmol/mouse VPAC2 agonist.

FIG. 6A shows the survival of BALB/c mice injected with 400%g of LPS at simultaneously, after 30 minutes, 1 or 4 hours, with 5 nmol of VIP, VPAC1 or VPAC2 agonist.

FIG. 6B shows the survival (%) versus LPS doses injected in mice treated with or without 5 nmol of VIP, VPAC1 or VPAC2 agonist.

FIGS. 7A & 7B show the production λ₁ of IL-6 by C57BL/6×129Sv or PAC1 −/−, respectively, mice peritoneal macrophages stimulated with 10 ng/ml LPS in the absence (control) or presence of 10⁻⁸M VIP or PACAP38.

FIG. 7C & 7D show the production of TNFα by C57BL/6×129Sv or PAC1−/−, respectively, mice peritoneal macrophages stimulated with 10 ng/ml LPS in the absence (control) or presence of 10⁻⁸M VIP or PACAP38.

FIG. 8 shows the circulating levels of TNFαand IL-6 in C57BL/6×129Sv or PAC1 −/− mice injected with 1 mg/mouse LPS, LPS plus 5 nmol/mouse VIP, or LPS plus 5 nmol/mouse PACAP38.

FIG. 9 shows the survival of C57BL/6×129Sv and PAC1 −/− mice injected with 1 mg of LPS with or without 5 nmol of VIP or PACAP38.

### DESCRIPTION OF THE PREFERRED EMBODIMENTS

This invention relates to the discovery of a role for VPAC1, VPAC2, and PAC1 receptor agonists in sepsis. These agonists have anti-inflammatory effects and inhibit the production of TNFα, IL-6, IL-12, IFNY, and NO, and also potenciate the production of the anti-inflammatory cytokine IL-10. These agonists can be administered in therapeutically effective doses to treat and/or prevent septic shock. The agonists of this invention typically are selected among several classes but preferably are analogs of VIP or PACAP.

As used herein, “Analogs of VIP or PACAP” means a peptide of the sequence of VIP or PACAP, or a fragment of them, or a peptide containing a part of these sequences with high affinity for VPAC1, VPAC2, or PAC1 receptors.

Further, as used herein, “Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution, liposomes. Besides the pharmaceutically acceptable carrier, the compositions of the invention can also comprise minor amounts of additives, such as stabilizers, excipients, buffers and preservatives.

The administration of such active ingredient may be by intravenous, intramuscular or subcutaneous route. Other routes of administration, which may establish the desired blood levels of the respective ingredients, are comprised by the present invention.

The active ingredients of the claimed compositions herein is a VPAC1, VPAC2, or PAC1 receptor agonist. Preferably, but not exclusively, they are polypeptides analogues of VIP or PACAP that bind VPAC1, VPAC2, or PAC1 receptors with high affinity.

VPAC1, VPAC2, or PAC1 receptor agonists can be administered to an individual in a variety of ways.

The routes of administration include intradermal, transdermal (e.g. in slow release formulations), intramuscular, intraperitoneal, intravenous, subcutaneous, oral, epidural, topical, and intranasal routes. In addition the VPAC1, VPAC2, or PAC1 receptor agonists can be administered together with other components of biologically active agents such as pharmaceutically acceptable surfactants, excipients, carriers, diluents and vehicles.

For parenteral (e.g. intravenous, subcutaneous, intramuscular) administration, VPAC1, VPAC2, or PAC1 receptor agonists can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The therapeutically effective amounts of a VPAC1, VPAC2, or PAC1 receptor agonist will be a function of many variables, including the type of agonist, the affinity of the agonist for its receptor, any residual cytotoxic activity exhibited by competitive agonists, the route of administration, the clinical condition of the patient.

Usually a daily dosage of active ingredient can be about 0.01 to 100 milligrams (mg) per kilogram of body weight. Ordinarily 1 to 100 milligrams per kilogram per day given in divided doses or in sustained release form is effective to obtain the desired results. Second or subsequent administrations can be performed at a dosage which is the same, less than or greater than the initial or previous dose administered to the individual. A second or subsequent administrations can be administered during or prior to relapse of the septic shock or the related symptoms. The terms “relapse” or “reoccurrence” are defined to encompass the appearance of one or more of symptoms of septic shock.

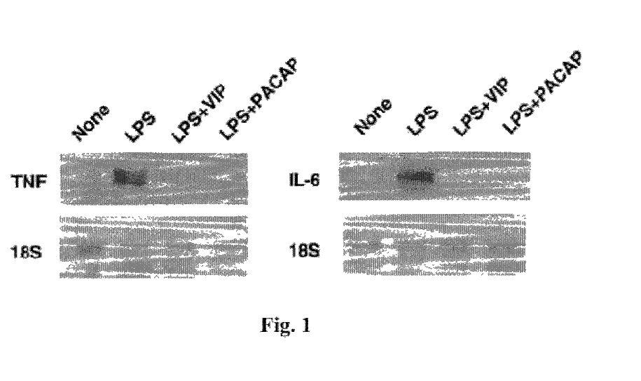

The present invention will now be illustrated by the following examples, which are not intended to be limiting in any way, and make reference to FIGS. 1-8.

### EXAMPLE 1

VIP and PACAP Inhibit the Transcription of TNFα and IL-6 in Macrophages from Mice Stimulated with LPS.

Method: BALB/c mice were injected with RPMI 1640 medium, LPS (400 μg), or LPS plus VIP or PACAP-38 (5 nmol). Peritoneal cells were harvested at 1 h (for TNFα) and 2 h (for IL-6), and total RNA was isolated from peritoneal cells. Twenty micrograms (jig) of total RNA from each sample were electrophoresed on 1.2% agarose-formaldehyde gel, transferred to nylon membranes, and cross-linked using UV light. Membranes were hybridized with specific probes for TNFα (5′-TTGACCTCAGCGCTGAGTTGGTCCCCCTTCTAGCTGGAAGACT-3′) and IL-6 (5′-CAAGAAGGCAACTGGATGAAGTCC TCTTGCAGAGAAGGAACTTCAT-3′) that were designed from the murine TNF-a and IL-6 cDNA published sequences (See, L. Fransen et al., Nucleic Acids Research 1985, 13:4417; and H. E. Grenett et al., Nucleic Acids Research 1990, 18:6455). The probe for the murine 18S RNA, as a quantity control for RNA, was an oligo-nucleotide (5′-CCAATTACAGGGCCTCGAAAGAGTCC TCTA-3′) derived from the published sequence. Oligonucleotides were 3′-labeled with digoxigenin-dUTP/dATP mix using terminal transferase, and hybridization and detection of chemoluminiscent signal were performed.

The results, as shown in FIG. 1, confirmed that VIP/PACAP significantly reduce the steady-state mRNA levels for both TNFα and IL-6 in peritoneal exudate cells. The results displayed in FIG. 1 were obtained by harvesting peritoneal cells at 1 h (for TNFα) and 2 h (for IL-6), and total RNA was prepared and subjected to Northern blot analysis using specific murine TNFα and IL-6 probes.

### EXAMPLE 2

VIP and PACAP reduce the levels of circulating TNF-αand IL-6 in mice treated with LPS.

Method: BALB/c mice were injected i.p. with LPS (400 μg) (control) or with LPS and VIP or PACAP-38 (5 nmol). Blood samples were taken at various time points by cardiac puncture (from 0 to 12 h). Blood samples were allowed to clot for 1 h at room temperature and serum was obtained and kept frozen. TNFα and IL-6 levels present in serum were determined using murine-specific sandwich ELISAs.

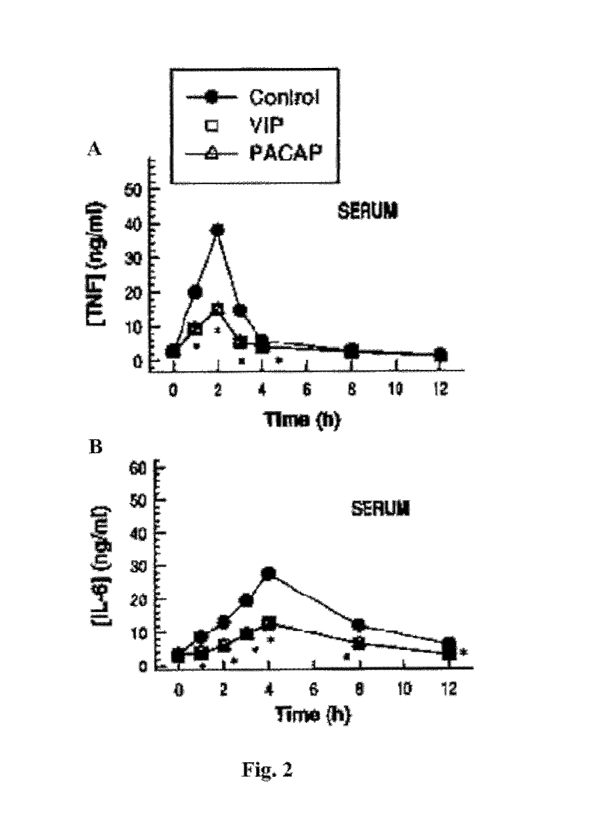

FIGS. 2A and 2B show the levels of TNFα and IL-6, respectively, in serum following injection with each of the control and two treatments. The control test points are indicated by circles, while the test points obtained from mice injected with VIP and PACAP are shown by squares and triangles, respectively. The results shown in FIGS. 2A and 2B show that both VIP and PACAP reduced by 50-60% the levels of secreted TNFα and IL-6 in serum at the peak of the response.

### EXAMPLE 3

VIP and PACAP Protect Against the Lethal Effects of LPS-Induced Septic shock.

Method: BALB/c mice were injected i.p. with LPS (400 μg) and VIP or PACAP-38 (5 nmol) at times 0, 30 min, 90 min or 4 h after LPS administration. Survival was monitored over the next 7 days. Survival curves were analyzed by the Kaplan-Meier method.

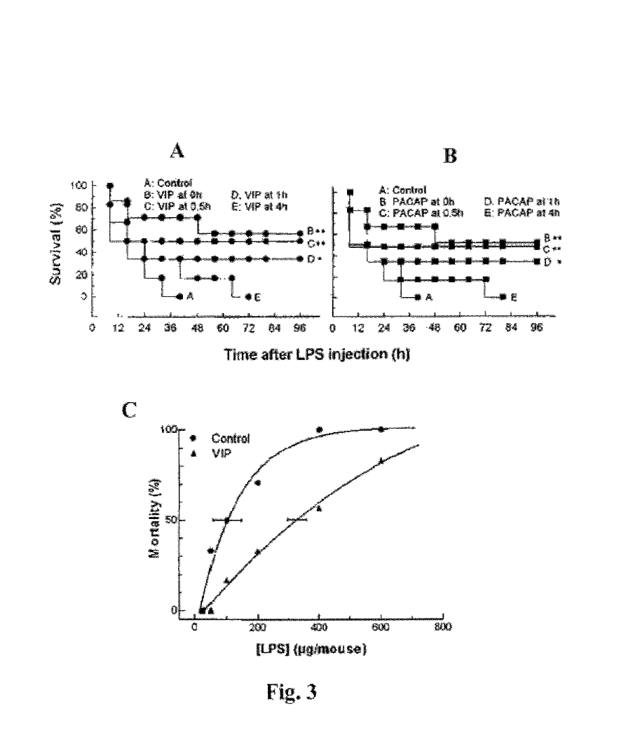

Results shown in FIGS. 3A and 3B show that mice injected with 5 nmol of VIP or PACAP and simultaneously with LPS have a survival rate near 60%. Full protective effect is exerted by VIP and PACAP even if given 1 h after LPS administration.

Also, BALB/c mice were injected i.p. with different concentrations (25-600 μg) of LPS, and survival was monitored over the next 4-7 days. Various doses of VIP were administered i.p. following injection of LPS. Control animals received only medium. Survival curves were used to calculate LD50.

The protective effect of VIP occurred over a large range of LPS concentrations, and VIP shifted the LD50 from 100 to 327 μg LPS, as shown in FIG. 3B. Test points from mice injected with VIP are shown by triangles, while control test points are shown by circles in FIG. 3B.

### EXAMPLE 4

VPAC1 and VPAC2 Agonists Reduce the Secretion of Proinflammatory Cytokines and NO Production and Stimulate the Secretion of IL-10 in Macrophages Stimulated with LPS.

Method: Purified macrophages were prepared from mice following i.p. injection of 2 ml 4% thioglycollate broth. After 4 days, the mice were killed, injected i.p. with 5 ml cold DMEM, followed by the harvesting of peritoneal fluid; the peritoneal exudate cells were washed and macrophages were obtained after the elimination of T and B cells through complement-mediated lysis following treatment with anti-Thy-1 and anti-B220 mAb.

The purified macrophage preparations were approximately 96% Mac-1 +by FACS analysis. Isolated peritoneal macrophages were seeded in flat-bottom 96-well microtiter plates at 8×10⁴ cells per well in a final volume of 200 μl DMEM supplemented with 2 mM L-glutamine, 100 U/ml penicillin, 10 μg/ml streptomycin and 10% FCS (complete DMEM) Cells were stimulated with 0.5 μg/ml of LPS in the absence or presence of different concentrations of VIP, VPAC1, VPAC2, or PAC1 agonists at 3720 C. in a humidified incubator with 5% CO2. [K 15,R 16,L 27 ]VIP [1-71]-GRF [8-27] was used as VPAC1-specific agonist (Gourlet P et al.; Peptides 1997,18:1539), Ro 25-1553 Ac-[Glu 8,Lys 12,Nle 17 ,Ala 19,Asp 25, Leu 26,Lys 27,28,Gly 29,30,Thr 31 ]-VIP cyclo [21-25] was used as VPAC2-specific agonist (See, M. Xia et al., The Journal of Pharmacology and Experimental Therapeutics 1997, 281:629). Cell-free supernatants were harvested at the designated time points and kept frozen (−20° C.) until cytokine and NO determination.

The amount of IL-6, TNFα, IL-10, and IL-12 in culture supernatants were determined by using specific sandwich ELISA. The amount of NO formed was stimated from the accumulation of the stable NO metabolite nitrite by the Griess assay (See, L. Green et al., Analytical Biochemistry 1982 126:131).

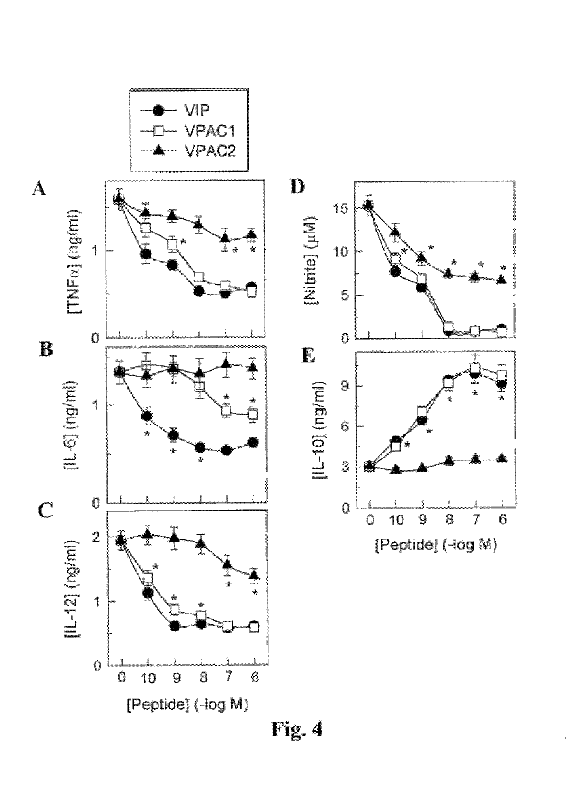

As shown in FIGS. 4A-4E, macrophages stimulated with LPS sequentially produced TNFα, IL-6, IL-12, NO and IL-10, respectively. In each graph of FIGS. 4A-4E, the production levels of the respective cultures after treatment with VIP (represented by circles), VPAC1 (squares) and VPAC2 (triangles) are illustrated.

Treatment of the cultures with various concentrations of the VPAC1 agonist significantly inhibited the production of TNFα, IL-12 and NO, and stimulated IL-10 secretion in a way similar to VIP. In addition, the VPAC1 agonist reduced IL-6 production, but its effect was lower than that of VIP, showing a significant inhibitory effect only at the highest concentrations used (10⁻⁷-10⁻⁶ M). On the other hand, whereas the VPAC2 agonist did not show any significant effect on IL-6 and IL-10 production, it slightly inhibited TNFα, IL-12 and NO production, although the inhibitory effects were much lower than those of the VPAC1-agonist.

### EXAMPLE 5

VPAC1 and VPAC2 agonists reduce the levels of circulating proinflammatory cytokines and NO, and augment IL-10 circulating levels in LPS-induced septic shock.

Methods: BALB/c mice were injected with 100 μg of LPS. Various doses of VIP, VPAC1 agonist VPAC2 agonist or PAC1 agonist were administered i.p. either concurrently with or following injection of LPS. [K 15, R 16,L 27] VIP [1-7]-GRF [8-27] was used as VPAC1-specific agonist, Ro 25-1553 Ac-[Glu 8, Lys 12, Nle 17, Ala 19, Asp 25, Leu 26, Lys 27,28, Gly 29,30, Thr 31 ]-VIP cyclo [21-25] was used as VPAC2-specific agonist. Control animals received only medium. Blood samples were taken at various time points by cardiac puncture (from 0 to 12 h). Blood samples were allowed to clot for 1 h at room temperature and serum was obtained and kept frozen. The amount of IL-6, TNFα, IL-10, and IL-12, and IFNY levels present in serum were determined using murine-specific sandwich ELISAS. The amount of NO was stimated from the accumulation of the stable NO metabolite nitrite by the Griess assay.

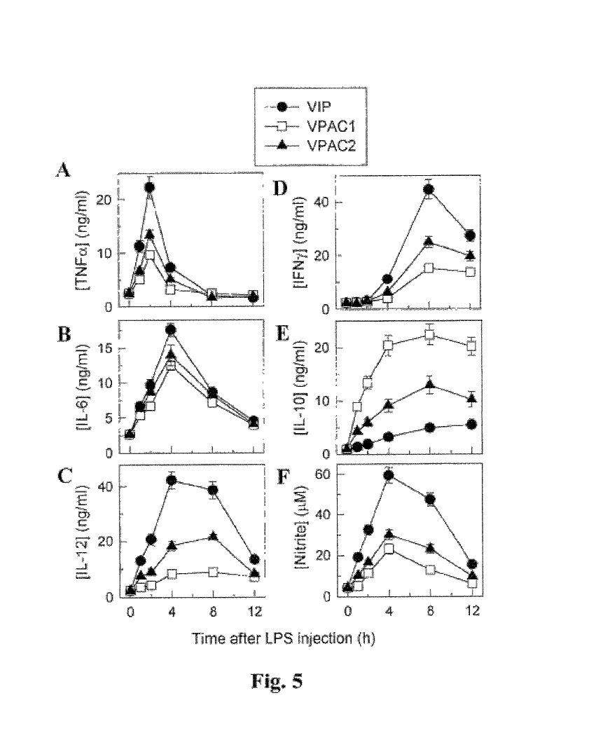

FIGS. 5A-5F show that, whereas both VPAC1 and VPAC2 agonists inhibited TNF-α levels in way similar to VIP, they had a much lower inhibitory effect on IL-6 levels. In each of FIGS. 5A-5F, the LPS-induced levels as affected by injections of VIP, VPAC1 and VPAC2 are shown by circles for VIP, open squares for VPAC1 and triangles for VPAC2.

Mice receiving VIP, VPAC1 or VPAC2 agonists in combination with LPS showed a significant reduction in serum IL-12 and IFNγ levels. Treatment of mice with VIP, VPAC1 or VPAC2 agonists significantly enhanced the LPS-induced IL-10 level in serum, and levels were still relatively high at 12 h. The VPAC2 agonist showed a much lower stimulatory activity in comparison with the VPAC1 agonist or VIP effect.

Finally, the peritoneal injection of LPS resulted in increased No amounts in serum, with peak at 4 h, and treatment with VIP or either VPAC agonists reduced NO. The in vivo effects of VIP and both VPAC1 and VPAC2 agonists on LPS-induced TNFα, IL-6, IL-12, IFNγ, IL-10 and NO were dose dependent, showing a maximum effect at 5-10 nmol.

### EXAMPLE 6

VPAC1, and VPAC2, Protect Against the Lethal Effects of LPS-Induced Septic Shock

Method: BALB/c mice were injected with different amounts (25-600 μg) of LPS, and survival was monitored over the next 4-7 days. Various doses of VIP, VPAC1 agonist VPAC2 agonist or PAC1 agonist were administered i.p. either concurrently with or following injection of LPS, at 30 min, 90 min or 4 h. [K 15, R 16, L 27]VIP[1-7]-GRF[8-27] was used as VPAC1-specific agonist, Ro 25-1553 Ac-[Glu 8, Lys 12, Nle 17, Ala 19, Asp 25, Leu 26, Lys 27,28, Gly 29,30, Thr 31]-VIP cyclo[21-25] was used as VPAC2-specific agonist. Control animals received only medium. Survival curves were analyzed by the Kaplan-Meier method.

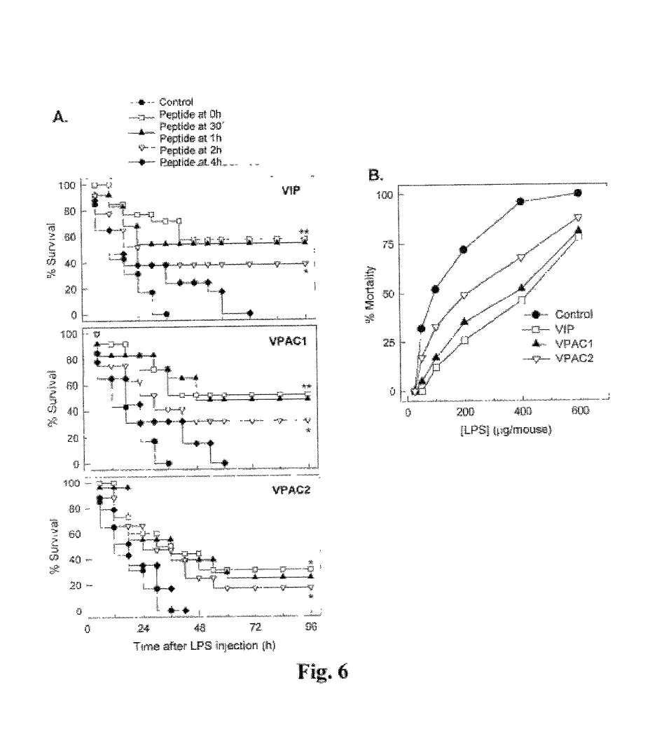

As indicated in FIG. 6A, VPAC1 and VPAC2 agonists significantly prevented endotoxin-induced death. In FIG. 6A, test points are represented by solid circles for the control group, open squares for concurrent injections of the peptide, solid triangles for injections given at 30 minutes, open triangles for injections given at 90 minutes and solid diamonds for injections given at 4 hours.

The VPAC1 agonist exhibited a potency similar to that of VIP (approximately a survival rate of 55%), whereas VPAC2 agonist was less efficient (30% survival). The effect of both agonists was dose dependent, with doses as low as 1 nmol being partially protective.

The protective effect of the VPAC1 and VPAC2 agonists occurred over a large range of LPS concentrations, and the VPAC1 agonist, similar to VIP, shifted the LD50 from 100 to 400 μg LPS, whereas the VPAC2 agonist shifted the LD50 to 225 μg LPS, as illustrated by FIG. 6B. In FIG. 6B, control test points are shown as circles, VIP test points are shown as open squares, VPAC1 test points are shown as solid triangles and VPAC2 test points are shown as open triangles.

Even for the nonsurvivors, VPAC2 agonists significantly increased, and VIP and the VPAC1 agonist almost doubled the time until death. Kinetic studies establish that, similar to VIP, both VPAC1 and VPAC2 agonists exerted a full protective effect when given up to 30 min after LPS administration, with a partial protection even at 90 min after shock induction.

### EXAMPLE 7

PAC1 Participates in the VIP/PACAP-Induced Protection from LPS-Induced Septic Shock.

Previous examples have shown that VIP and PACAP attenuate the deleterious consequences of septic shock acting through VPAC1 and VPAC2 receptors.

We have used mice deficient in PAC1 receptor (knock-out for PAC1) to elucidate the role of this receptor in the protective role of PAC1 receptor agonists in endotoxic shock.

Method: Adult male and female mice deficient in PAC1 receptor (PAC1 −/−) were obtained by gene targeting (See, F. Jamen et al., The Journal of Clinical Investigation 2000, 105:1305). PAC1 −/− mice were compared with wild type (C57BL/6×129Sv) counterparts, which serve as controls.

Macrophages elicited for 4 d with thioglicollate or resident macrophages were obtained by peritoneal lavage using 4 ml of RPMI 1640 medium. Peritoneal exudate cells were washed and resuspended in ice-cold medium supplemented with 2% heat-activated fetal calf serum containing β-ME, amino acids, penicillin, and streptomicyn. Cells were plated in 96-well tissue cultureplates at 8×10 4 cells per well in a final volume of 0.2 ml in duplicate. After 2 h at 37° C. in 5% CO2, nonadherent cells were removed by repeated washing.

At least 96% of the adherent cells were macrophages as judged by morphological and phagocytic criteria and by flow cytometry. Macrophage monolayers were incubated in RPMI 1640 complete medium and stimulated with different concentrations of LPS (10 ng/ml) in the presence or absence of VIP or PACAP38 (from 10⁻¹² to 10⁻⁷ M) at 37° C. in a humidified incubator with 5% CO². Cell free supernatants were harvested at the designated time points and kept frozen (−20° C.) until assayed for IL-6 and TNFα production. The amount of IL-6 and TNFα present in supernatants was determined by using specific sandwich ELISA.

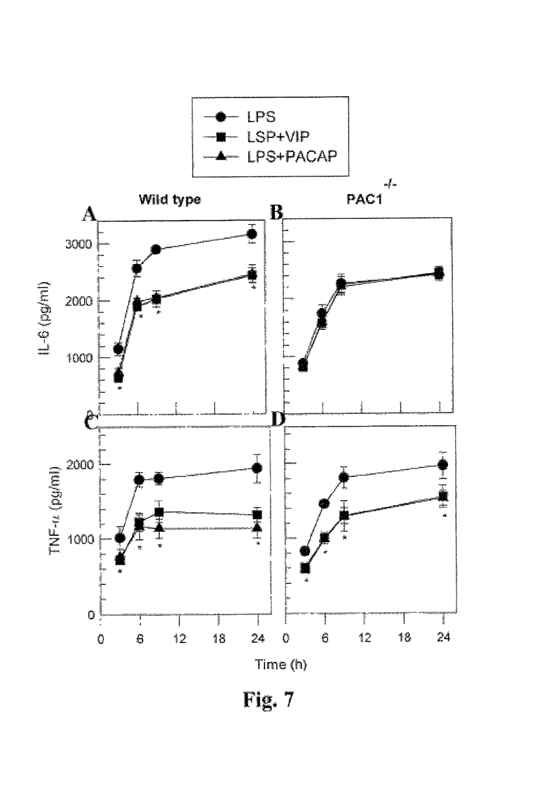

FIGS. 7A-7D illustrate the levels of IL-6 (FIGS. 7A & 7B) and TNFα (FIGS. 7C & 7D) in wild type and PAC1 −/− mice, respectively, assayed following treatment with VIP and PACAP compared to a control group. Test points for mice injected with LPS only (control group) are shown by circles, while those for mice injected with LPS and VIP are represented by squares and mice injected with LPS and PACAP are shown by triangles.

VIP and PACAP38 inhibited in a time dependent manner IL-6 and TNFα production in LPS stimulated macrophages from wild type mice, as shown by FIGS. 7A-7D. In contrast, whereas VIP/PACAP significantly diminished, in a dose-dependent manner, endotoxin-induced TNFα levels from PAC1 −/− mice at all times assayed, both peptides failed to inhibit IL-6 production.

Mice receiving LPS (1 mg) alone or mice injected with LPS concurrently with VIP or PACAP were sacrificed after various time points. Blood was extracted by puncture and the blood samples were allowed to clot for 1 h at room temperature; serum was obtained and kept frozen until TNFα and IL-6 measurement by ELISA.

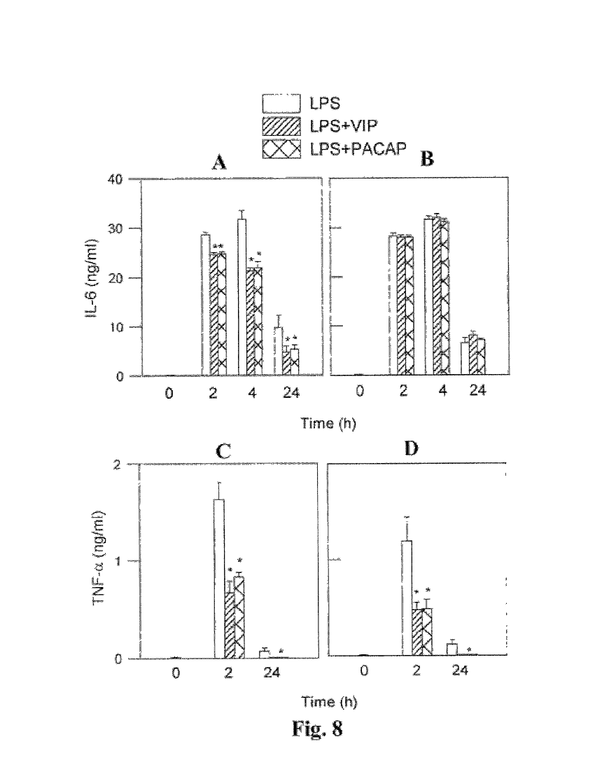

FIGS. 8A-8D show the results of the ELISA measurements, with the LPS only injection control group levels represented by open bars, levels for mice injected with both LPS and VIP represented by diagonal-line shaded bars, and levels for mice injected with LPS and PACAP shown by cross-hatched shaded bars.

FIGS. 8A & 8B graphically illustrate how IL-6 levels increased slowly and remained elevated long after LPS injection in both types of mice, with a peak at 4 h. FIG. 8A shows that in wild type mice, VIP/PACAP treatment resulted in a inhibition of 30% on IL-6 levels circulating levels. However, the production of this cytokine was not affected by either peptide in PAC1 −/− mice, as seen in FIG. 8B.

FIGS. 8C & 8D shows the levels of TNFα in mice.

A sharp increase in the TNFα levels was observed within 2 h of the LPS injection in both mice types.

The addition of VIP or PACAP resulted in a reduction on the levels of circulating TNFα, with maximum values of 60%.

Wild type and PAC1−/− mice were injected i.p. with 1 mg of LPS and survival was monitored over the next 4-7 days. A 5 nmol dose of VIP or PACAP38 was administered i.p. concurrently with LPS injection. Control animals received only medium. Survival curves were analyzed by the Kaplan-Meier method.

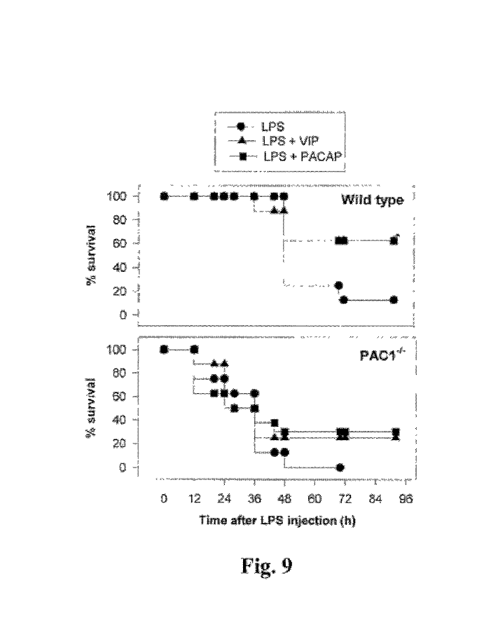

In wild type mice, both VIP and PACAP38 protected against the lethal effect of LPS with a survival rate of 60%. However, the injection of either two peptides to PAC1 −/− mice only prevented death around 25%, as seen in FIG. 9. Survival time was twice as long in wild type in comparison to PAC1 −/− mice.

While a specific embodiment of the invention has been shown and described in detail to illustrate the application of the principles of the invention, it will be understood that the invention may be embodied otherwise without departing from such principles.

What is claimed is:

1. A pharmaceutical composition for the treatment and/or prevention of septic shock, comprising VPAC1 receptor agonist with a pharmaceutically acceptable carrier.

2. A pharmaceutical composition for the treatment and/or prevention of septic shock, comprising VPAC2 receptor agonist with a pharmaceutically acceptable carrier.

3. A pharmaceutical composition for the treatment and/or prevention of septic shock, comprising PAC1 receptor agonist with a pharmaceutically acceptable carrier.

4. A method for treating and/or preventing septic shock, comprising administering a therapeutically effective dose of a VPAC1 receptor agonist to a patient.

5. A method for treating and/or preventing septic shock comprising administering a therapeutically effective dose of a VPAC2 receptor agonist to a patient.

6. A method for treating and/or preventing septic shock comprising administering a therapeutically effective dose of a PAC1 receptor agonist to a patient.


SEQUENCE LISTING (3 sequences)

SEQ ID NO: 1 - protein, 28 aa, unknown
  Isolated from small intestines and brains of pigs
HSDAVFTDNYTRLRKQMAVKKYLNSILN

SEQ ID NO: 2 - protein, 38 aa, unknown
  Isolated from ovine hypothalmi or rat brains
HSDGIFTDSYSRYRKQMAVKKYLAAVLGKRYKQRVKNK

SEQ ID NO: 3 - protein, 27 aa, unknown
  Isolated from ovine hypothalmi or rat brains
HSDGIFTDSYSRYRKQMAVKKYLAAVL